(19) United States
(12) Patent Application Publication
(10) Pub. No.: US 2026/0286238 A1
(43) Pub. Date: Sep. 24, 2026

(54) COUPLED SUPERCRITICAL FLUID - FISCHER-TROPSCH INPUT APPARATUS AND METHOD OF USE THEREOF

(71) Applicants: Alton Reich, Huntsville, AL (US); Christopher Jackson, San Juan, PR (US); Duchuy Huynh, Labadieville, LA (US)

(72) Inventors: Alton Reich, Huntsville, AL (US); Christopher Jackson, San Juan, PR (US); Duchuy Huynh, Labadieville, LA (US)

(21) Appl. No.: 19/094,866

(22) Filed: Mar. 29, 2025

Related U.S. Application Data
(63) Continuation-in-part of application No. 19/093,797, filed on Mar. 28, 2025.
Application No. 19/093,797 is a continuation-in-part of application No. 19/093,617, filed on Mar. 28, 2025.
Application No. 19/093,617 is a continuation-in-part of application No. 19/088,431, filed on Mar. 24, 2025.
Application No. 19/088,431 is a continuation-in-part of application No. 19/088,018, filed on Mar. 24, 2025.
Application No. 19/088,018 is a continuation-in-part of application No. 19/087,486, filed on Mar. 22, 2025.

(51) Int. Cl. C10J 3/46; C07C 1/04

(52) U.S. Cl. CPC C10J 3/463; C07C 1/0495; C10J 2300/0946

(57) ABSTRACT

The invention comprises a method for processing waste material with a supercritical fluid to produce a Fischer-Tropsch process input, comprising the steps of breaking down the waste material in the supercritical fluid in a supercritical fluid chamber and incorporating at least supercritical water in the supercritical fluid, optionally including any of the steps of: maintaining a pressure of greater than two hundred seventeen bar in the supercritical fluid chamber; monitoring at least one breakdown process of the waste material with light from a spectrometer; transferring into an input chamber of the Fischer-Tropsch process an auxiliary hydrogen gas from a non-supercritical fluid source; feeding back into the supercritical fluid chamber at least one byproduct generated in the Fischer-Tropsch system; and generating a fuel, in the Fischer-Tropsch system incorporating the Fischer-Tropsch process, with at least a twelve carbon aliphatic.

### CROSS-REFERENCES TO RELATED APPLICATIONS

This application is a continuation-in-part of U.S. patent application Ser. No. 19/093,737 filed Mar. 28, 2025, which is a continuation-in-part of U.S. patent application Ser. No. 19/093,617 filed Mar. 28, 2025, which is a continuation-in-part of U.S. patent application Ser. No. 19/088,431 filed Mar. 24, 2025, which is a continuation-in-part of U.S. patent application Ser. No. 19/088,018 filed Mar. 24, 2025, which is a continuation-in-part of U.S. patent application Ser. No. 19/087,486 filed Mar. 22, 2025, all of which are incorporated herein in their entirety by this reference thereto.

### BACKGROUND OF THE INVENTION

### Field of the Invention

The invention relates generally to a supercritical fluid system used to generate fuel from waste feedstocks.

### Discussion of the Prior Art

### Problem

There exists in the art a need to recycle waste products into a usable product, such as a fuel.

### SUMMARY OF THE INVENTION

The invention comprises a supercritical fluid apparatus/system and method of use thereof for recycling waste products into useful substances.

### DESCRIPTION OF THE FIGURES

A more complete understanding of the present invention is derived by referring to the detailed description and claims when considered in connection with the Figures, wherein like reference numbers refer to similar items throughout the Figures.

FIG. 1 illustrates a recycling system;

FIG. 2 a system for generating fuel from a waste feedstock;

FIG. 3 illustrates feedstocks;

FIG. 4 illustrates waste materials as a feedstock;

FIG. 5 illustrates food crops as a feedstock;

FIG. 6 illustrates non-food crops as a feedstock;

FIG. 7 illustrates oilseed as a feedstock;

FIG. 8 illustrates fossil fuels as a feedstock;

FIG. 9 illustrates a sensor system;

FIG. 10 illustrates hydrogen feedback into a supercritical fluid system;

FIG. 11 illustrates Fischer-Tropsch sensors;

FIG. 12 illustrates Fischer-Tropsch feedback into a supercritical fluid system;

FIG. 13 illustrates a Fischer-Tropsch sensing system;

FIG. 14 illustrates electrolysis coupled to a supercritical fluid system;

FIG. 15 illustrates carbon capture linked to a supercritical fluid system;

FIG. 16A and FIG. 16B each illustrate monitoring a supercritical fluid chamber;

FIG. 17 illustrates monitoring a supercritical fluid system;

FIG. 18 illustrates a molecular hydrogen phase diagram;

FIG. 19 illustrates a water phase diagram;

FIG. 20 illustrates a first feedback control loop; and

FIG. 21 illustrates a Fischer-Tropsch input control system.

Elements and steps in the figures are illustrated for simplicity and clarity and have not necessarily been rendered according to any particular sequence. For example, steps that are performed concurrently or in different order are illustrated in the figures to help improve understanding of embodiments of the present invention.

### DETAILED DESCRIPTION OF THE INVENTION

The invention comprises a method for processing waste material with a supercritical fluid to produce a Fischer-Tropsch process input, comprising the steps of:
breaking down the waste material in the supercritical fluid in a supercritical fluid chamber; and
incorporating at least supercritical water in the supercritical fluid, with optionally and preferably one or more of the steps of:
   maintaining a pressure of greater than two hundred seventeen bar in the supercritical fluid chamber;
   monitoring at least one breakdown process of the waste material with light from a spectrometer penetrating into the supercritical fluid chamber;
   spectroscopically sensing a carbon monoxide quantity that is input into the input chamber of a Fischer-Tropsch system, at least a portion of the carbon monoxide quantity comprising the carbon monoxide output from the supercritical fluid chamber;
   non-spectroscopically determining a molecular hydrogen quantity that is input into the input chamber of the Fischer-Tropsch system, at least a portion of the molecular hydrogen quantity comprising the molecular hydrogen output from the supercritical fluid chamber;
   transferring into an input chamber of the Fischer-Tropsch process both carbon monoxide and molecular hydrogen formed in the step of breaking down and an auxiliary hydrogen gas from a non-supercritical fluid source;
   feeding back into the supercritical fluid chamber at least one byproduct generated in the Fischer-Tropsch system; and
   generating a fuel in the Fischer-Tropsch system with at least a portion of the carbon monoxide and at least a portion of the molecular hydrogen, the fuel comprising a twelve carbon aliphatic component of both kerosene and diesel.

### Overview

Generally, a recycling system is presented that converts waste feedstock into a useful product, such as a synthesis gas, hydrogen gas, and ultimately to produce a fuel, such as a synthetic fuel from the synthesis gas. The recycling system is optionally a source of basic chemicals, such as sulfur.

The recycling system has multiple optional interlinked systems, such as: (1) a fuel generation system using a Fischer-Tropsch system linked to output of a supercritical fluid system; (2) a hydrogen feedback loop into the supercritical fluid system; (3) a light hydrocarbon feedback loop from the Fischer-Tropsch system into the supercritical fluid system; (4) use of heated water from the supercritical fluid system as an input to an electrolysis system generating hydrogen gas; and/or (5) a carbon sequestration system linked to the Fisher-Tropsch system, each of which are further described, infra.

### Fuel Generation From Feedstock

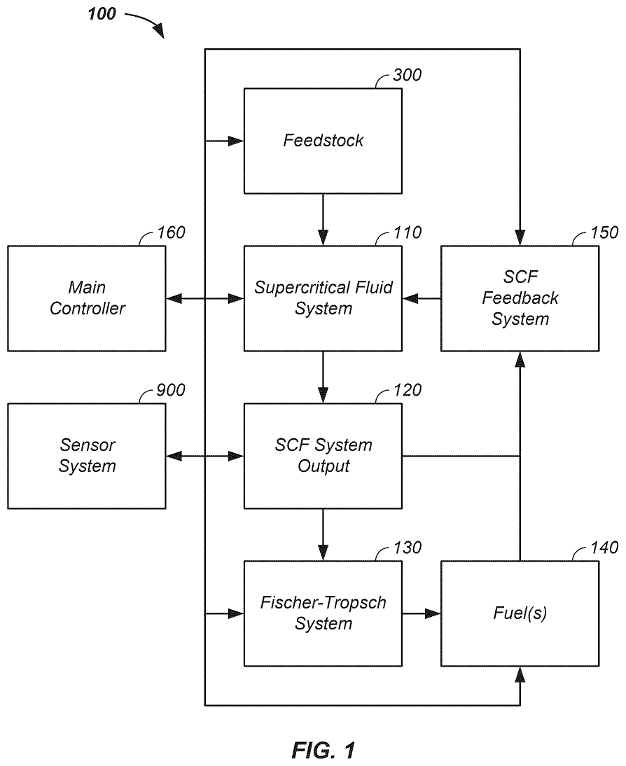

Referring now to FIG. 1, a recycling system 100 is illustrated. Generally, a feedstock 300, such as one or more waste materials, is used as an input to a supercritical fluid system 110. The supercritical fluid system 110 generates, with a high pressure and a high temperature therein, a supercritical fluid system output 120 comprising small molecules, such as synthesis gas, that are appropriate for use as an input to a Fischer-Tropsch system 130, where the Fischer-Tropsch system is used to generate a fuel 140, such as a synthetic fuel and/or an aviation fuel. The above process is generally referred to as the above described fuel generation system using a Fischer-Tropsch system linked to a supercritical fluid system. Optionally and preferably, the supercritical fluid system output 120 and/or an output of the Fischer-Tropsch system 130 is used as a feedback into a supercritical fluid feedback system 150 as an input to the supercritical fluid system 110.

Still referring to FIG. 1, herein a Fischer-Tropsch process is any collection of chemical reactions that converts a mixture of carbon monoxide and hydrogen gas, known as syngas, into liquid hydrocarbons. Further, the Fischer-Tropsch system 130 herein is a system that includes any implementation of the Fischer-Tropsch process. Stated again, the Fischer-Tropsch system 130 optionally and preferably uses any process/processes of converting converts a mixture of carbon monoxide and hydrogen gas into a fuel, such as a liquid hydrocarbon. The Fischer-Tropsch system 130 optionally and preferably includes controls/sensors coupled to input from output from the supercritical fluid system 110, such as the supercritical fluid system output 120, which contains at least carbon monoxide and hydrogen gas.

Still referring to FIG. 1, a main controller 160 is used to control any one or more elements of the recycling system 100, such as through use of sensor readings from a sensor system 900, which is also linked to any one or more elements of the recycling system 100. The main controller 160 and the recycling system 900 are each further described, infra. Herein a feedback controller is optionally the main controller 160 or a sub-system of the main controller 160.

### Synthetic Fuel Generation/Production

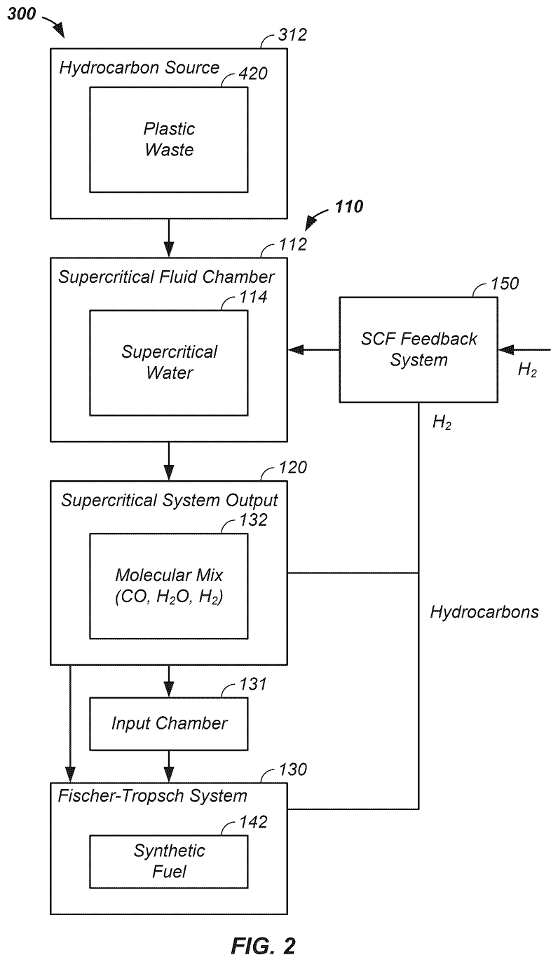

Referring now to FIG. 2, for clarity of presentation and without loss of generality an example of generation of a fuel/synthetic fuel from a hydrocarbon source is provided. Generally, in this non-limiting example, the feedstock 300 is illustrated as a hydrocarbon source 312, where the hydrocarbon source comprises at least carbon bonded to hydrogen, and optionally and preferably comprises a feedstock material that is at least 50, 60, 70, 80, 90, 95, 98, or 99% carbon-hydrogen bonds by mass, such as waste material comprising plastic waste 420. In this non-limiting example, the plastic waste 420 is prepared in any fashion, such as grinding, as an input to a supercritical fluid chamber 112 of the supercritical fluid system 110. The supercritical fluid chamber 112 is configured to hold any supercritical fluid, such as a supercritical water 114 and/or a supercritical carbon dioxide. A supercritical system output 120 from the supercritical fluid chamber 112 is optionally a small molecular weight compound, such as a molecular mix 132 of water, H₂O, and/or synthesis gas, such as carbon monoxide, CO, and/or molecular hydrogen, H₂. The supercritical fluid chamber 112 is optionally and preferably maintained at a desired temperature, such as great than −240, −100, 0, 50, 100, 250, or 500° C. and/or above a pressure of greater than 13, 50, 100, 150, 200, or 217 bar, with a temperature regulator affixed to a temperature sensor and/or a heater and/or with a pressure regulator coupled with a pressure sensor and compression/decompression pump, which are not illustrated for clarity of presentation. Other outputs such as sulfur, a form or sulfur, and/or waste are additionally optionally generated. In this non-limiting example, at least one component of the molecular mix 132, such as the synthesis gas, is used as an input to the Fischer-Tropsch system 130, where the Fischer-Tropsch system 132 is used to generate the fuel 140, such as a synthetic fuel 142. Herein, a synthesis gas comprises carbon monoxide and hydrogen and optionally comprises methane and carbon dioxide. Herein, optionally and preferably at least 25, 50, 60, 70, 80, 90, or 95% of sulfur in the feedstock 300 in the supercritical system output 120 is in elemental form and/or as a non-oxygenated sulfur form. A transfer system is optionally and preferably used to move any product/byproduct out of the supercritical fluid system, such as to a waste system, a carbon capture system, into a Fischer-Tropsch system, to an electrolysis system, and/or to a supercritical fluid system. The transfer system is optionally any mechanical and/or electromechanical system and/or any material/mass transfer system known in the mechanical arts.

Still referring to FIG. 2, the Fischer-Tropsch system 130 is further described. The Fischer-Tropsch system optionally and preferably contains a series of reactions occurring in a series of connected systems to generate the fuel 140, which is in some examples of the system a set of procedures. However, an input to the Fischer-Tropsch system is simply an input chamber 131 or set of input chambers, such as configured to receive into the Fischer-Tropsch system starting materials, such as carbon monoxide and molecular hydrogen. Thus, output from the supercritical fluid system 110 is optionally thought of as merely being transferred to the input chamber of the Fischer-Tropsch system 130 without requiring all or indeed any elements of the Fischer-Tropsch system 130, which might already be in existence. Further, the Fischer-Tropsch system 130 as a system generates an output of fuel 140. Thus, the input chamber 131 and the output fuel 140 are optionally and preferably considered herein to be outside of the Fischer-Tropsch system 130, such as inputs into and outputs from the Fischer-Tropsch system 130, respectively. Thus, in one embodiment of the invention, the supercritical fluid system 110 yields a molecular mix 132 of a supercritical system output 120 including at least carbon monoxide and molecular hydrogen that is provided to an input chamber 131 linked to a Fischer-Tropsch system 130 without requiring additional elements of any known Fischer-Tropsch process, where a Fischer-Tropsch process is accepted shorthand in the field to those skilled in the art that refers to any process/processes of converting carbon monoxide and molecular hydrogen, with optional additional inserts and/or controls, to form fuel.

Still referring to FIG. 2, typical optional monitored breakdown products / intermediate products, such as from plastic, in a supercritical fluid system include one or more hydrocarbons, one or more alcohols, and/or one or more ketones. Examples include: ethylene glycol, terephthalic acid or dimethyl terephthalate, Styrene, hydrochloric acid, chlorinated hydrocarbons, monomers, toluene, benzene, alkanes, alkenes, waxes, light oils, bisphenol-A, and/or dimethyl carbonate, where desired end products are the above described hydrogen molecule and carbon monoxide.

Still referring to FIG. 2, the Fischer-Tropsch system 130/Fischer-Tropsch process, in its simplest view, is a collection of chemical reactions configured to convert a mixture of carbon monoxide and hydrogen into liquid hydrocarbons, which is the fuel 140. The Fischer-Tropsch reactions optionally and preferably occur: in the presence of metal catalysts, at temperatures of 150 to 300° C. (302 to 572° F.), and/or at pressures of one to several tens of atmospheres. The Fischer-Tropsch system 130 is further described, infra.

Still referring to FIG. 2, a portion of the supercritical system output 120, such as excess hydrogen; byproducts of the Fischer-Tropsch system, such as light weight hydrocarbons and/or hydrogen gas; and/or additional inputs, such as hydrogen gas, are optionally and preferably fed into/fed back into the supercritical fluid chamber 112, such as through the supercritical feedback system 150. The supercritical fluid feedback system 150 is further described, infra.

### Feedstock

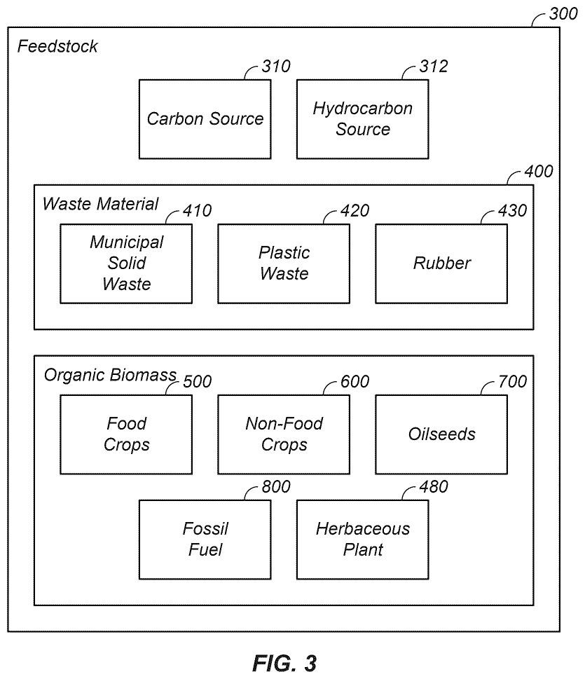

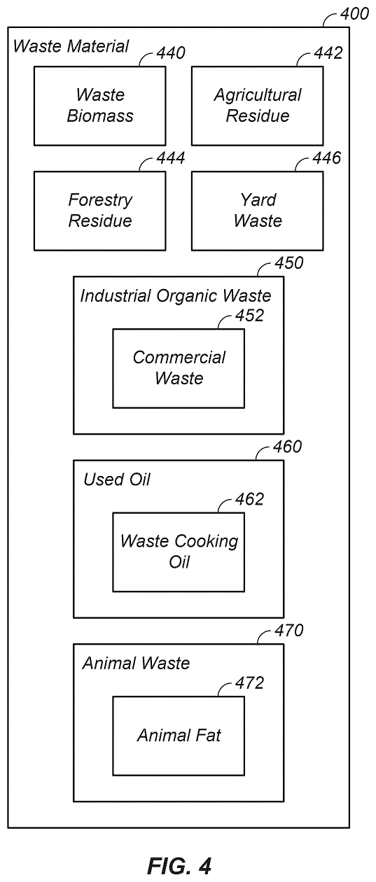

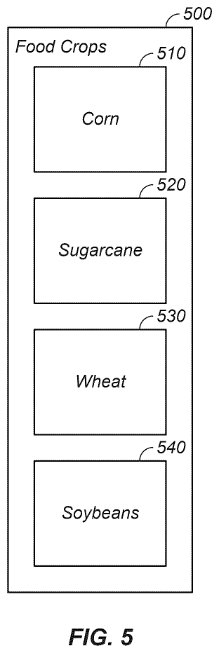

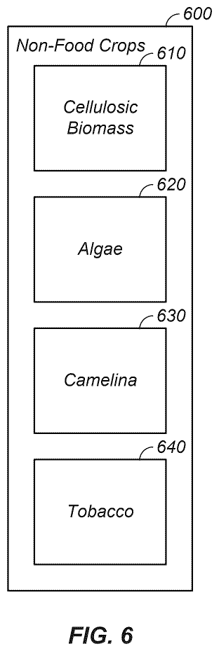

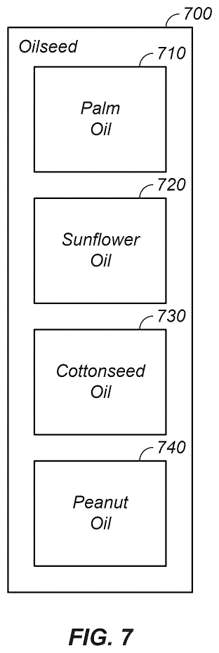

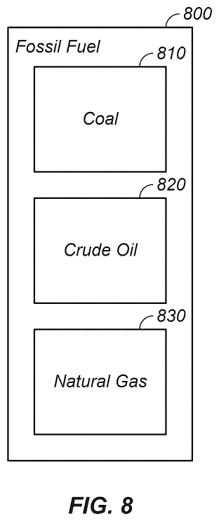

Referring now to FIGS. 3-8, the feedstock 300, used as input to the supercritical fluid system 110, is further described. Generally, as one goal of the overall system is recycling, preferred feedstocks are waste products. However, generally, any one or more feedstocks are optionally used/blended in any ratio in the current system. For instance, a carbon source 310 is optionally used as the feedstock 300, where the carbon source is greater than 25, 50, 75, or 95% carbon by mass. Similarly, a hydrocarbon source 312 is optionally used as the feedstock 300, where forms of carbon and forms of hydrogen comprise greater than 25, 50, 60, 70, 80, or 90 of the feedstock 300 by mass. Similarly, a waste material 400 is optionally used as the feedstock 300, where the waste material comprises greater than 25, 50, 75, 80, 90, or 95% of the mass of the feedstock 300. For clarity of presentation and without loss of generality, a few waste materials 400, such as municipal solid waste 410, plastic waste 420, and/or rubber 430, are used herein as examples feedstocks 300. Other examples of feedstocks include: (A) other waste materials 400, such as a waste biomass 440, an agricultural residue 442, a forestry residue 444, yard waste 446, an industrial organic waste 450, such as commercial waste 452; a used oil 460, such as a waste cooking oil 462; and/or an animal waste 470, such as animal fat 472 or beef tallow; (B) a food crop 500, such as corn 510, sugarcane 520, wheat 530, soybeans 540, sugar beets, and/or any food; (C) a non-food crop 600, such as a cellulosic biomass 610, an algae 620, Camelina 630, tobacco 640, or any non-food crop; (D) an oilseed 700, such as palm oil 710, sunflower oil 720, cottonseed oil 730, peanut oil, 740, or any soil from any seed; (D) a fossil fuel 800, such as coal 810, crude oil 820, natural gas 830, and/or any mined/mining derived fuel; and/or (E) a herbaceous plant 480, where any percentage mix of the waste product sources are used, such as under control of the main controller 160 to increase efficiency of the system, such as the Fischer-Tropsch system 130 in generation of a given/selected fuel 140/fuel type.

### Sensor System

As described supra, the sensor system 900 optionally and preferably operates in conjunction with the main controller 160 to monitor any one or more systems/subsystems/processes/sub-processes/elements of the recycling system 100 and/or any input and/or output of the recycling system 100. For clarity of presentation and without loss of generality, seven exemplary sensor systems/sensors examples are described here.

### EXAMPLE I

In a first example, a feedstock input sensor 910/set of sensors, such as a feedstock chemical input sensor 912, a feedstock physical input sensor 914, and/or a feedstock rate of input sensor 916, used individually and/or in combination, measure any element/property of the feedstock 300. For instance, the feedstock chemical input sensor(s) 912 measure with any technology amounts of chemical types/chemical classes of the feedstock 300, such as amount of carbon, hydrogen, or any element, molecule, or chemical class in any state. Similarly, the feedstock physical input sensor(s) 914 measure, with any technology, any physical property/physical state of the feedstock 300, such as temperature of the feedstock 300 and/or particle size/grind size/filter size of the feedstock 300. The feedstock rate of input sensor(s) 916 measure flow of the feedstock stream or one or more of several feedstock streams 916 into the supercritical fluid system 110, where each sensor optionally and preferably provides input directly and/or indirectly to the main controller 160, where the main controller controls any chemical reaction, physical state, heater, and/or flow of any one or more elements of the recycling system 100. The main controller 160 is optionally provided additional inputs, such as market price for products, current needs, and/or manually entered and/or computer fed input directions/requests.

### EXAMPLE II

In a second example, a supercritical fluid chamber sensor 920/set of sensors is used to monitor any temperature 922 and/or pressure 924 of the supercritical fluid chamber itself and/or a content/content state of the supercritical fluid chamber 112. Optionally, any chemical sensor and/or any physical sensor (not illustrated for clarity of presentation), such as described in the first example of this section is optionally used to measure chemical breakdown and/or physical state of anything contained in the supercritical fluid chamber 112, such as a state of breakdown of the feedstock 300 in the supercritical fluid chamber 112. Optionally, residence time 926 is recorded, monitored, and/or measured for any component in the supercritical fluid chamber 112. The supercritical fluid chamber sensors are further described, infra.

### EXAMPLE III

In a third example, supercritical fluid system output sensors 930 are described. Generally, a supercritical chemical output sensor 932 functions like the feedstock chemical input sensor 912, but detects breakdown products of the feedstock 300 fed through the supercritical fluid chamber 112 into an output stream/substance. Exemplary monitored output substances comprise: carbon monoxide, water, hydrogen, sulfur compounds, and/or any breakdown product/molecular mix 132 output from the supercritical fluid chamber, where elements of the output comprise elements of synthesis gas. The supercritical fluid physical output sensor 934 measures any physical property of the output stream, such as temperature, pressure, particle size. The supercritical fluid waste output sensor 936 measures any property of waste generated in the supercritical fluid chamber 112. For example, knowledge of detected generated excess waste output is fed to the main controller 160 to allow for adjustments of the input rate of the feedstock 300 to the supercritical fluid chamber 112, and/or any one of more of the temperature 922, the pressure 924, and/or the residence time 926 of components of the feedstock 300 in the supercritical fluid chamber 112 to increase yield of fuel 140, increase efficiency, and/or to minimize waste from the recycling system 100.

### EXAMPLE IV

In a fourth example, a feedback input sensor 940, also referred to as feedback input sensors 940 and/or at least one feedback sensor, are used to monitor/sense any element of the supercritical feedback system 150, where the supercritical feedback system 150 comprises at least a chamber, a compartment, a tube, and/or a tunnel configured to feed a substance into the supercritical fluid system 110 and/or the supercritical fluid chamber 112 thereof. For instance, a feedback chemical input sensor 942 measures any chemical property of any feedback element entering, passing through, and/or exiting the supercritical feedback system 150, such as a quantity/state of hydrogen and/or a quantity/state of a short chain hydrocarbon, such as methane, ethane, propane, butane, and/or pentane. Herein, a short-chain hydrocarbon comprises a molecule comprising carbon chains with less than 16, 14, 12, 10, 8, or 6 carbons per chain, on average for a sampling of the feedback substance. The feedback physical input sensor(s) 944 measure any physical property/state of components entering, passing through, and/or exiting the supercritical feedback system 150, such as temperature, volume, concentration, and/or state. The feedback rate of input sensor 946 measures flow and/or quantity of components moved as a function of time out of the supercritical feedback system 150 into the supercritical fluid system 110.

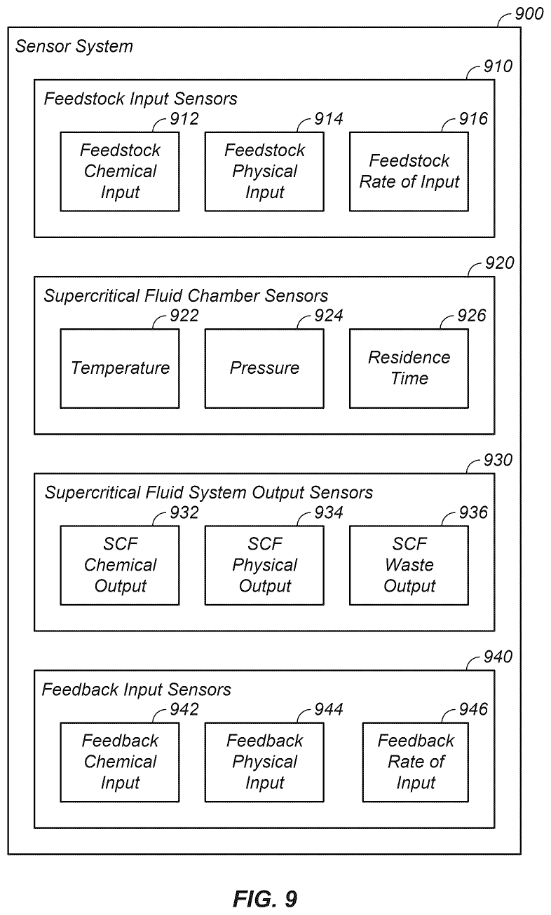

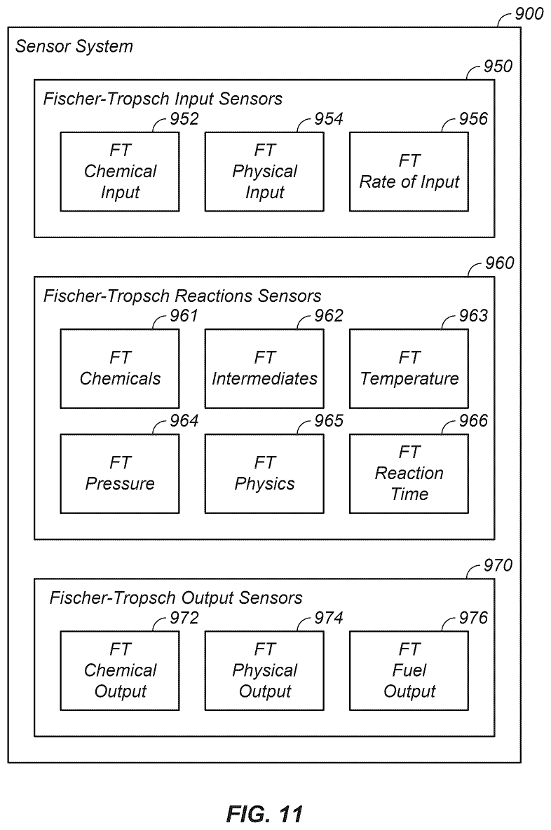

Referring still to FIG. 9, the above described examples of sensors in the sensor system 900 monitor state of the supercritical fluid system 110 along with inputs and outputs thereof. Referring still to FIG. 9 and referring now to FIG. 11, in the following examples, examples of sensors in the sensor system 900 monitor aspects of the Fischer-Tropsch system 130. Again, sensors in the sensor system 900 coordinate control of the recycling system 100 and/or any element therein via the main controller 160.

### EXAMPLE V

In a fifth example, Fischer-Tropsch input sensors 950 are described. Fischer-Tropsch chemical input sensors 950 measure synthesis gas components, such as carbon monoxide and molecular hydrogen and/or the supercritical fluid system output 120, described infra. The Fischer-Tropsch physical input sensors 954 measure state of the synthesis gas components, such as temperature and pressure, while the Fischer-Tropsch rate of input sensors 956 measure flow rate and/or quantity of input components, such as synthesis gas components.

### EXAMPLE VI

In a sixth example, Fischer-Tropsch reaction sensors 960 are described. Fischer-Tropsch chemical sensors 961 measure chemical state of the synthesis gas components and the Fischer-Tropsch intermediate sensors 962 measure methane, the state of growth of short chain hydrocarbons, and/or long chain hydrocarbons, such as the fuel 140, along with any one or more of the reaction processes in the formation of the methane, growing longer chain hydrocarbons, and/or byproducts/waste. Fischer-Tropsch temperature sensors 963 measure temperatures of the various reactions/containers in the Fischer-Tropsch system 130. Fischer-Tropsch pressure sensors 964 measure pressures in the various reactions/containers in the Fischer-Tropsch system 130. Fischer-Tropsch physics sensors 965 measure any physical property, stirring, separation, and/or filtering of the starting materials, intermediates, products, and/or waste material in the Fischer-Tropsch system 130. Fischer-Tropsch time sensors 966 monitor any reaction time, transit time, movement, and/or lag time of any constituent passing through various chambers/processes in the Fischer-Tropsch 130. As with all sensors herein, results are optionally and preferably fed to the control system, where they are used to monitor/enhance the recycling system 100. Again, the Fischer-Tropsch system 130/process is further described infra.

### EXAMPLE VII

In a seventh example, Fischer-Tropsch output sensors 970 are described. Fischer-Tropsch chemical output sensors 972 measure output fuel 140 and/or by-products such as hydrogen, methane, and short chain hydrocarbons output from the Fischer-Tropsch system 130. Fischer-Tropsch physical output sensors 974 measure any physical property of components/mixtures output from the Fischer-Tropsch system 130, such as density, viscosity, purity, and/or quantity. Fischer-Tropsch fuel output sensors 976 measure physical properties of fuel 140 generated in the Fischer-Tropsch system, such as quantity, purity, density, and type.

### EXAMPLE VIII

In an eighth example, hydrogen gas is measured using a supercritical fluid chemical output sensor 932 comprising any one or more of an electrochemical sensors, gas chromatography, and/or a thermal conductivity detector configured to measure a concentration and/or generate a signal related to a quantity of hydrogen gas. Optionally, the electrochemical sensors, the gas chromatography system, and/or the thermal conductivity detector are referred to herein collectively as non-spectroscopic sensors. Non-spectroscopic sensors for sensing hydrogen gas are preferred as hydrogen gas does not have a substantive molecular vibration in the mid-infrared, near-infrared, and/or visible range due to the balanced nature of the hydrogen-hydrogen bond in molecular hydrogen. Thus, the non-spectroscopic sensor is optionally referred to as a non-visible and/or non-infrared sensor and non-light based sensor.

### EXAMPLE IX

In a ninth example, any sensor and/or controller described herein is optionally used to measure an input quantity, an input flow rate, and/or an input mass/concentration of any chemical component described herein into the input chamber 131 of a Fischer-Tropsch process and/or the Fischer-Tropsch system, such as a carbon monoxide quantity/mass and/or a molecular hydrogen quantity/mass.

Generally, any of the above described sensors in the sensor system 900 provide input/feedback to the main controller 160, which allows the main controller 160 to alter any aspect of the recycling process, such as feedstock type, feedstock rate, feedstock mix, flow rate, temperature, pressure, reaction times, mixing, auxiliary input quantity/rate, and/or feedback through the feedback system 150 to enhance efficiency, increase output, minimize waste, optimize value, and/or meet demand.

### Supercritical Fluid Feedback System

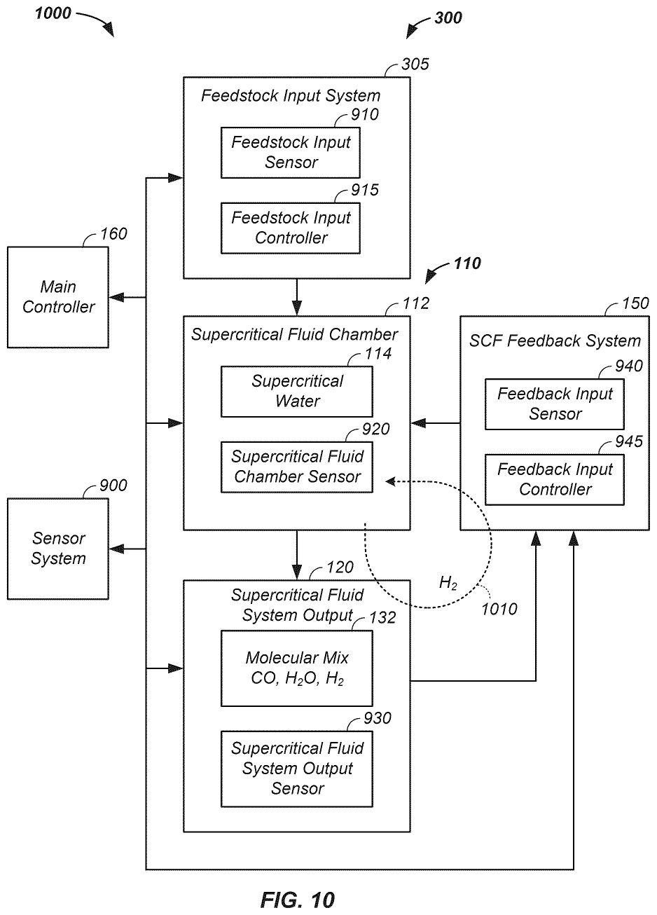

Referring now to FIG. 10, a supercritical fluid feedback system 1000 is described. For clarity of presentation and without loss of generality, the system is described by way of example. Generally, a feedstock input system of the feedstock system 300 uses the feedstock input sensor(s) 910 and a feedstock input controller 915, such as under control of the main controller 160 to input a feedstock into the supercritical fluid chamber 112 of the supercritical fluid system 110. In this non-limiting example, supercritical water 114 and conditions thereof, such as further described infra, are used to breakdown the molecularly complex feedstock into more basic components, such as molecular hydrogen, carbon dioxide, water, and the like as described supra. The supercritical fluid chamber sensors 920 optionally and preferably monitor the breakdown process and/or are used in a control loop with the main controller 160 to continue, alter, and/or end the breakdown process, such as based on a yield or percentage of breakdown products achieved. For instance, breakdown is stopped once greater than 25, 50, 60, 70, 80, 90, or 95 percent of molecules, by number, have a molecular weight less than 250, 200, 150, 100, 50, 37 (such as CO), 19 (such as H₂O), or 17 (such as CH₄). In another example, breakdown is stopped when formation of a monitored molecule/substance slows to an accepted threshold. For instance, carbon monoxide will form at a rate that eventually slows, indicative of the breakdown process coming to an end as the feedstock/material has been processed. The slowed rate of formation of carbon monoxide, or any monitored breakdown product, is optionally and preferably used as a signal to a supercritical fluid chamber controller, such as linked to the main controller, to end the breakdown process and/or as a start time, in a batch system, of starting to remove components from the supercritical fluid chamber 112. Stated again, supercritical fluid system output 120, such as the molecular mix 132 of carbon monoxide, molecular hydrogen, carbon dioxide, and hydrogen gas, as described supra, is output and optionally and preferably monitored with the supercritical fluid system output sensors 930. Optionally and preferably, the sensors are spectroscopic sensors, as further described infra.

In an optional and preferable hydrogen feedback system 1010, molecular hydrogen, generated as a component of the supercritical fluid system output 120 is fed back into the supercritical fluid system 110, such as through the supercritical fluid feedback system 150. Optionally and preferably, the supercritical fluid feedback system 150 is monitored with the feedback input sensors 940 and/or controlled with a feedback input controller 945. Stated again, excess molecular hydrogen, such as not needed by stoichiometric ratios of the Fischer-Tropsch system is optionally and preferably fed back into the supercritical fluid chamber 110 in the hydrogen feedback system 1010. Optionally supplemental molecular hydrogen is fed into the supercritical fluid system 110, such as through the supercritical fluid feedback system 150, as illustrated in FIG. 2.

### Supercritical Fluid-Fischer-Tropsch Coupled Fuel System

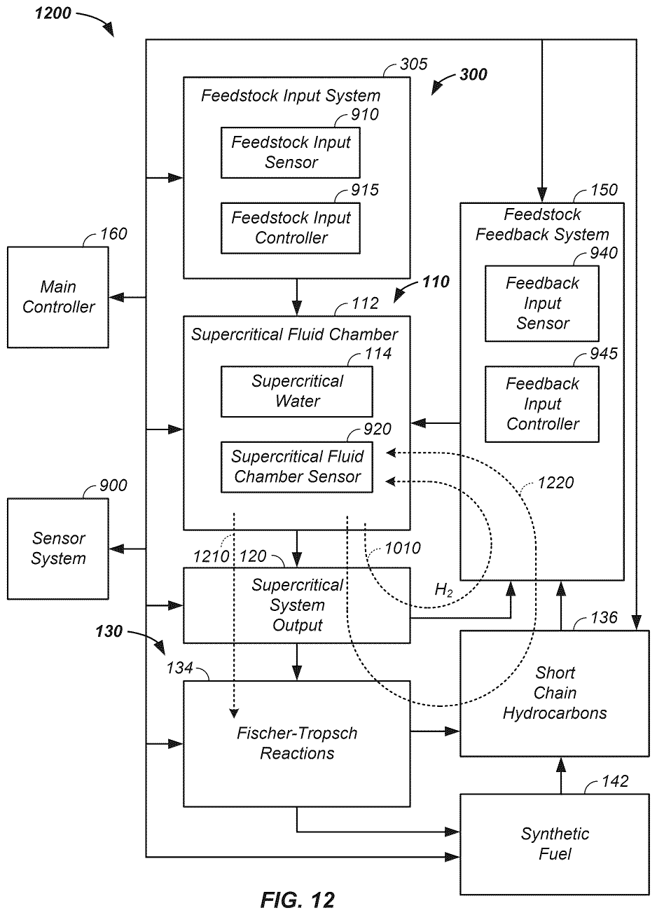

Referring now to FIG. 12, a supercritical fluid-Fischer-Tropsch coupled system 1200 is illustrated. In this example provided for clarity of presentation and without loss of generality, generally, any one or more elements of the supercritical fluid feedback system 1000 is included in a supercritical fluid-Fischer-Tropsch coupled fuel production system 1210. However, in the supercritical fluid-Fischer-Tropsch fuel production system 1210, at least a portion of the supercritical system output 120 is provided as an input to the Fischer-Tropsch system 130, as further described supra.

Still referring to FIG. 12, in the supercritical fluid-Fischer-Tropsch fuel production system 1210 at least some of the supercritical system output 120, such as at least some of the molecular hydrogen and at least some of the carbon monoxide generated in the supercritical fluid chamber 112 is provided as an input into the Fischer-Tropsch system 130, such as for use in Fischer-Tropsch reactions 134, which generate at least fuel 140, illustrated here as synthetic fuel 142. Further, auxiliary inputs, such as carbon monoxide and/or hydrogen gas, are optionally used to supplement inputs described herein, such as output from the supercritical fluid system 110. An overview of the Fischer-Tropsch system 110 is provided here.

### Fischer-Tropsch System/Process

Generally, details of the Fischer-Tropsch processes are known. However, herein, the Fischer-Tropsch system 130 couples elements of the Fischer-Tropsch processes with the main controller 160 and/or the sensor system 900 in the fuel generation system 100 using the supercritical fluid system 100. As such, only basics of the Fischer-Tropsch process are provided herein for clarity of presentation and without loss of generality.

In a typical implementation of the Fischer-Tropsch process, carbon monoxide and hydrogen, the feedstocks for Fischer-Tropsch process, are produced from coal, natural gas, or biomass in a process known as gasification. The Fischer-Tropsch process converts the carbon monoxide and molecular hydrogen into synthetic lubrication oil and/or synthetic fuel. Notably, in the above described use of the supercritical fluid system 110, elemental sulfur or solid sulfur, S₍ₛ₎, is optionally and preferably separated out as a waste product, which results in reduced SOₓ input into the Fischer-Tropsch system 110 and a corresponding production of low sulfur content fuel. Similarly, nitrogen is separated out as nitrogen gas, N_{2(g)}, in the supercritical fluid system 110 and nitrous oxide output is optionally and preferably controlled to less than 25, 15, 5, 4, 3, 2, 1, or 0.5% of total nitrogen output from the supercritical fluid chamber 112, which avoids creation of NOₓ greenhouse gases. The Fischer-Tropsch process involves a series of chemical reactions that produce a variety of hydrocarbons, ideally having the formula (CₙH₂ₙ₊₂). The more useful reactions produce alkanes according to equation 1, where n is typically 10-20.

(2n+1) H₂+n CO→CₙH₂ₙ₊₂+n H₂O (eq. 1)

The formation of methane (n=1) is unwanted. Most of the alkanes produced in the Fischer-Tropsch process are straight-chain molecules, which are suitable as a diesel fuel. In addition to alkane formation, competing reactions give small amounts of alkenes, as well as alcohols and other oxygenated hydrocarbons, any of which are optionally fed back into the supercritical fluid system 110, such as via the feedstock feedback system 150.

Generally, converting a mixture of molecular hydrogen, H₂, and carbon monoxide, CO, into aliphatic products is a multi-step reaction with several intermediate compounds. The growth of the hydrocarbon chain is optionally visualized as involving a repeated sequence in which hydrogen atoms are added to carbon and oxygen, such as where the C-O bond in carbon monoxide is split and a new C—C bond in the growing hydrocarbon is formed. For one —CH2— group produced by CO+2 H₂→(CH₂)+H₂O, several reactions are are typically performed:
associative adsorption of CO;
splitting of the C—O bond;
dissociative adsorption of 2 H₂
transfer of 2 H to the oxygen to yield H₂O;
desorption of H₂O; and
transfer of 2 H to the carbon to yield CH₂.

Stated again, the conversion of carbon monoxide to alkanes involves hydrogenation of the carbon monoxide, the hydrogenolysis (cleavage with H₂) of C—O bonds, and the formation of C—C bonds. Referring again to FIG. 2, in the recycling system 100 described herein, hydrocarbons in the feedstock 300, such as from the municipal solid waste 410, the plastic waste 420, and/or the rubber 430, as processed by the supercritical fluid system 110, are used to generate the starting components of carbon monoxide and hydrogen for the Fischer-Tropsch system 130.

Again, generally in the supercritical fluid-Fischer-Tropsch coupled system 1200, output of the supercritical fluid system 110 is used as an input to the Fischer-Tropsch system 130 used to generate fuel 140. For instance, hydrocarbons from the feedstock, such as plastics, yield at least carbon monoxide and molecular hydrogen.

### Fischer-Tropsch Feedback System

Referring again to FIG. 12, another example of the supercritical fluid-Fischer-Tropsch coupled system 1200 is described. Again, generally, any one or more elements of the supercritical fluid feedback system 1000 and/or the supercritical fluid-Fischer-Tropsch fuel production system 1210 is included in a Fischer-Tropsch feedback system 1220. Generally, the Fischer-Tropsch feedback system 1220 includes a Fischer-Tropsch feedback loop from the Fischer-Tropsch system 130 into the supercritical fluid system 110, as further described supra.

In the Fischer-Tropsch feedback system 1220, Fischer-Tropsch reactions 134 generate both: (1) the fuel 140, such as the synthetic fuel 142, and (2) by-products, such as alcohols, aldehydes, ketones, acids, and short chain hydrocarbons 136. While any component generated by the Fischer-Tropsch reactions 134 is optionally fed back into the supercritical fluid system 100, for clarity of presentation and without loss of generality only the short chain hydrocarbons 136 are illustrated in the Fischer-Tropsch feedback system 1220 herein. More particularly, the short-chain hydrocarbons 136 are illustrated as being fed from the Fischer-Tropsch system 130 into the feedstock feedback system 150, where they are optionally and preferably monitored with the feedback input sensor 940 and/or controlled by the feedback input controller 945 before being transported/moved back into the supercritical fluid chamber 112. Recycling of Fischer-Tropsch byproducts reduces waste, reduces input requirements, and/or enhances efficiency of use of the supercritical fluid system 110 and/or any element of the recycling system 100.

Optionally and preferably, the byproducts and/or the short chain hydrocarbons 136 are maintained at a temperature of greater than 80, 100, 120, 150, or 180 degrees Fahrenheit during at least one and optionally all of the steps of: generation of the byproduct in the Fischer-Tropsch reactions 135, transfer of the byproduct into the feedstock feedback system 150, monitoring the byproduct, controlling movement of the byproduct, and/or feeding the byproduct back into the supercritical fluid chamber 112, which avoids energy requirements of reheating an originally heated product that was allowed to cool before feeding the byproduct into the supercritical fluid chamber.

Any one or more elements of the supercritical fluid-Fischer-Tropsch fuel production system 1210, the hydrogen feedback system 1010, and the Fischer-Tropsch feedback system 1220 are optionally run in any order, in series, and/or in parallel.

### Fischer-Tropsch Sensing System

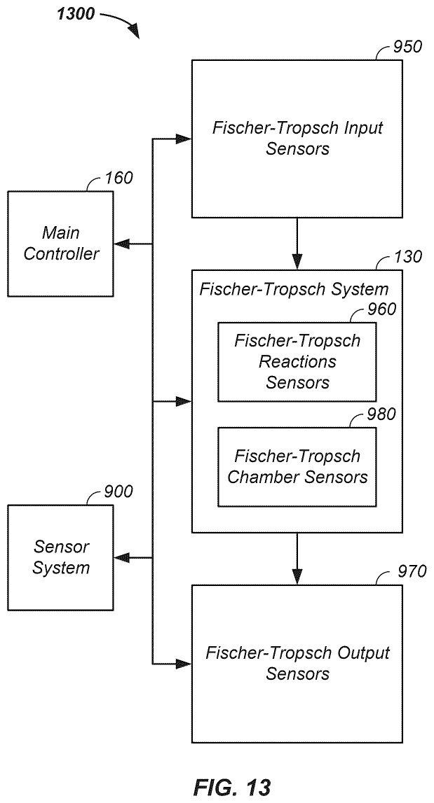

Referring now to FIG. 13, an optional Fischer-Tropsch sensing system 1300 is illustrated. Generally, optional and preferable Fischer-Tropsch input sensors 950 monitor any chemical and/or physical element of components fed into the Fischer-Tropsch system 130. Similarly, optional and preferable Fischer-Tropsch output sensors 970 monitor any chemical and/or physical product and/or byproduct exiting the Fischer-Tropsch system 130. Further, the Fischer-Tropsch system 130 itself optionally and preferably uses Fischer-Tropsch reaction sensors 960 to measure progression of the fuel generation process, as a feedback to the main controller 160 controlling the Fischer-Tropsch processes and/or uses Fischer-Tropsch chamber sensors 980 to monitor state of the one or more Fischer-Tropsch chambers and/or elements therein, such as any chemical and/or physical property and/or flow rate of components therein.

### High Temperature Water-Electrolysis Coupled System

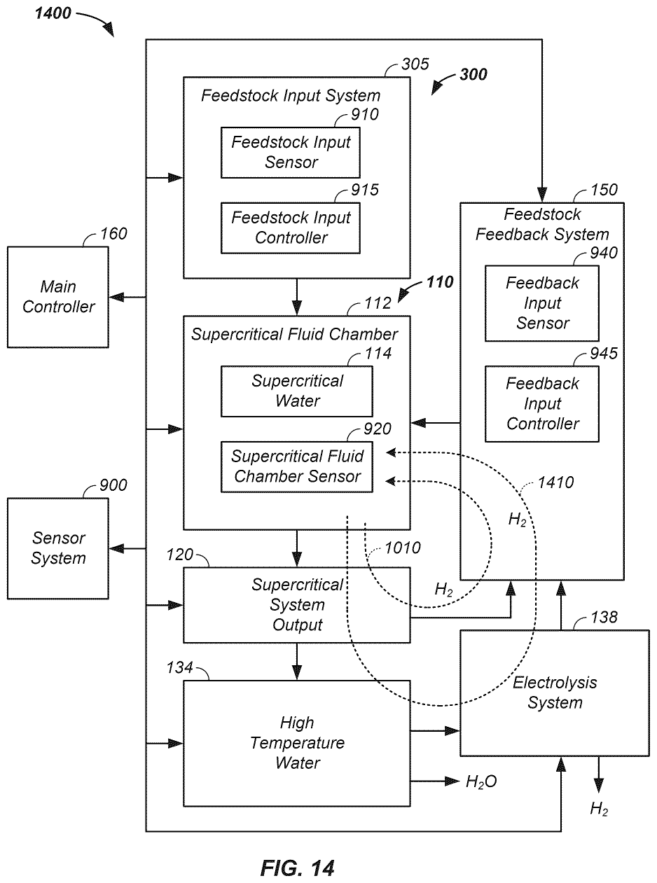

Referring now to FIG. 14, an optional supercritical fluid generated high temperature water-electrolysis coupled system 1400 is illustrated. Generally, the supercritical fluid system 110 generates as an output high temperature water 134. The high temperature water 134, which is filtered and clean, is: (1) optionally output for other uses and/or (2) is used as an input to an electrolysis system 138. The electrolysis system 138 optionally and preferably generates molecular hydrogen, which is fed back into the supercritical fluid chamber 112, such as through the feedstock feedback system 150 in a high temperature water electrolysis feedback system 1410. The feedback input sensor 940, feedback input controller 945, and/or the main controller 160 optionally and preferably time and/or control the amount of the hydrogen fed back to the supercritical fluid chamber 112 in the high temperature water-electrolysis feedback system 1410. An optional sensor and/or controller is a temperature controller, such as linked to a temperature sensor, where the temperature controller is linked to one or more elements of the high temperature water-electrolysis feedback system 1410 to ensure that the high temperature water remains above a threshold, such as greater than 90, 100, 120, or 150 degrees Fahrenheit in the system and/or is assisted by a heater to remain at an elevated temperature of greater than 90, 100, 120, or 150 degrees Fahrenheit in the system. A key benefit is that the water is already heated as an input to the electrolysis system, which reduces cost of molecular hydrogen formation. Generally, any known electrolysis process that produces hydrogen is used in the electrolysis system 138.

### Carbon Capture System

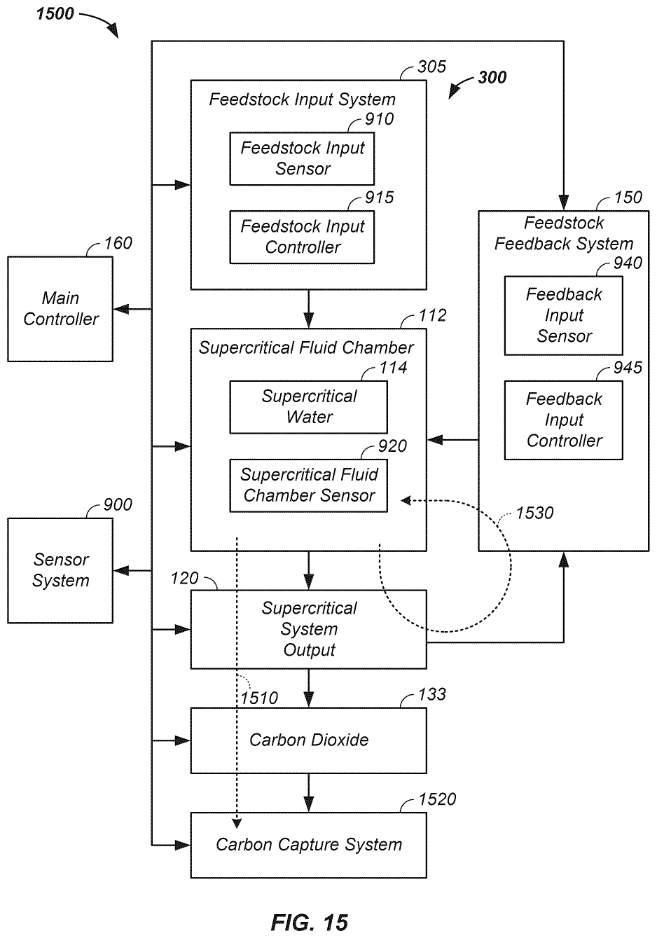

Referring now to FIG. 15, an optional carbon capture system 1500/carbon sequestration is illustrated. As illustrated, in this example, a carbon capture path 1510 takes carbon dioxide output from the supercritical fluid system 110 via the supercritical system output 120 and provides it as an input to a carbon capture system 1520. Generally, any known carbon capture process that captures carbon dioxide is optionally used in the carbon capture system 1520. Again, advantages include the purity of the produced carbon dioxide, the heated condition of the carbon dioxide, the proximity of the carbon dioxide source, and the capture of the carbon dioxide before release to the environment.

Still referring to FIG. 15, a feedback loop 1530 optionally includes any one or more elements of the above described: hydrogen feedback system 1010, the Fischer-Tropsch feedback system 1220, and/or the high temperature water electrolysis feedback system 1410.

### Pathways

Referring again to FIGS. 12, 14, and 15, any one or more elements of the supercritical fluid-Fischer-Tropsch fuel production system 1210, the hydrogen feedback system 1010, the Fischer-Tropsch feedback system 1220, the high temperature water-electrolysis feedback system 410, and/or the carbon capture path 1510 are optionally run in any order, in series, and/or in parallel.

### Supercritical Fluid System Monitoring

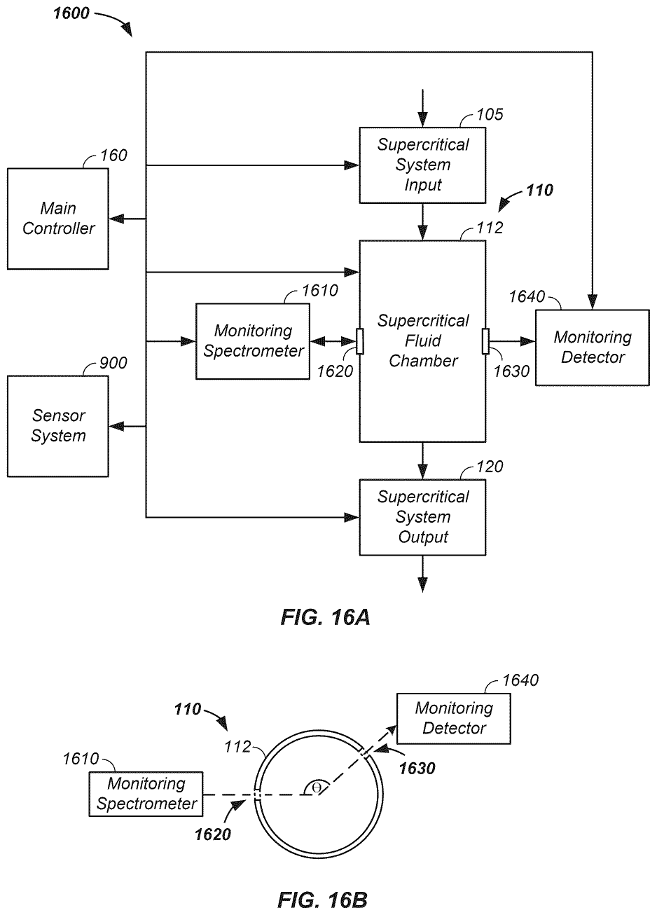

Referring now to FIG. 16A and FIG. 16B, an optional supercritical fluid system monitoring system 1600 is illustrated, which is optionally a stand-alone system for use with any supercritical fluid system and/or is optionally an element of the recycling system 100. Generally, a supercritical system input 105, such as from the feedstock 300, provides an input to a supercritical fluid chamber 112, such as in a supercritical fluid system 110, and a supercritical system 120 is generated, such as fed into the Fischer-Tropsch system 130. Monitoring is further described here.

Referring still to FIG. 16A, in the supercritical fluid monitoring system 1600, at least one monitoring spectrometer 1610 is used to monitor any element of the supercritical system input 105, the supercritical fluid chamber 112, and/or the supercritical system output 120. In this example, provided for clarity of presentation and without loss of generality, the monitoring spectrometer 1610 is any type of spectrometer/analyzer, such as: an ultraviolet spectrometer, such as operating in a range of 200 to 400 nm; a visible wavelength spectrometer; a near infrared spectrometer, such as operating in a range of 700 to 1100 nm and/or 1100 to 2500 nm; a mid-infrared spectrometer, such as operating in a range of 4000 to 700 cm⁻¹; a far infrared spectrometer; an X-ray system; a radio wave system; and/or a gamma ray system. Examples of the monitoring spectrometer include a Raman spectrometer and/or a spectrometer incorporating use of a tunable diode laser. Optionally a residual gas analyzer and/or a mass spectrometer is used in place of and/or with any of the spectrometers described herein. As illustrated, the monitoring system optionally and preferably sends signals/waves to/from the supercritical fluid chamber 112 through a first window 1620, such as an optical window, a quartz window, and/or a sapphire window. Generally, a monitoring detector 1640 of the monitoring spectrometer 1610 is positioned in the monitoring spectrometer, such as for detection of a reflective and/or diffusely reflected signal and/or is positioned at any angle along an incident light path, such as (as illustrated) in an optional position opposite the supercritical fluid chamber 112, relative to the monitoring spectrometer 1610 and/or a source thereof. Optionally, reflected, transmitted, absorbed and/or altered wavelengths/signals pass through a second window 1630 with any of the properties of the first window. Generally, any range of wavelengths presented herein includes any sub-range of wavelengths thereof. Referring now to FIG. 16B, an angle, Θ, between incident light and detected light is illustrated at any optional angle from 0 to 360 degrees, such as from 1 to 180 degrees. Further, the supercritical fluid chamber 112 is optionally of any geometry, such as with an entrance side/wall and an exit side/wall, even when the supercritical fluid chamber has a rounded/curved wall.

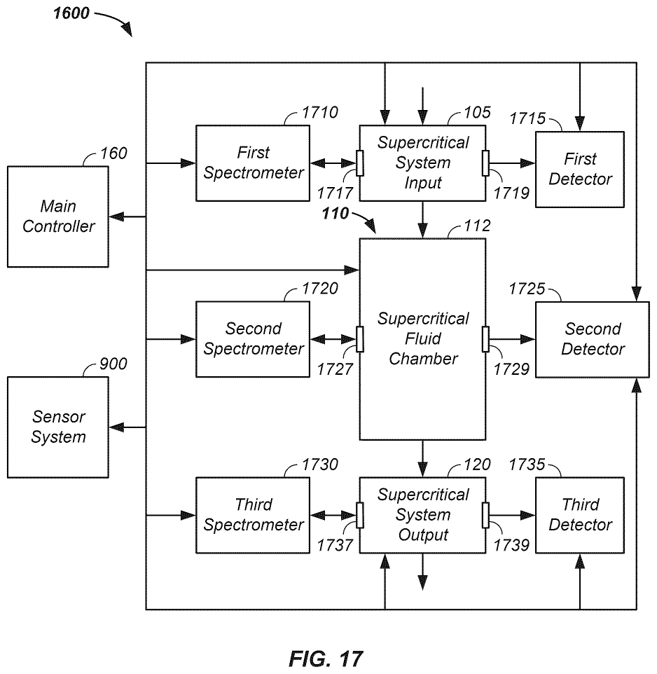

Referring still to FIG. 16A and referring now to FIG. 17, the supercritical fluid monitoring system 1600 is further described. Generally, the monitoring spectrometer 1610/spectrometer system is optionally a set of spectrometers. For clarity of presentation and without loss of generality, the monitoring spectrometer 1610/spectrometer system is illustrated in this example as: (1) a first spectrometer 1710 coupled with a first input window 1717 to a first detector 1715 via a first output window 1719 in a system configured to analyze any aspect of the supercritical system input 105/input system and/or elements thereof; (2) a second spectrometer 1720 coupled with a second input window 1727 to a second detector 1725 via a second output window 1729 in a system configured to analyze any aspect of the supercritical fluid chamber 112 and/or elements thereof; and/or (3) a third spectrometer 1730 coupled with a third input window 1737 to a third detector 1735 via a third output window 1739 in a system configured to analyze any aspect of the supercritical system output 120 and/or elements thereof, where any of spectrometers are optionally and preferably elements of the sensor system 900 and/or are linked to the main controller 160. Herein, the terminology of the first spectrometer, second spectrometer, and third spectrometer are optionally and preferably used to refer to any step of monitoring a processed material. To further clarify, as illustrated, the first spectrometer is illustrated as monitoring an initial material, the second spectrometer is used to measure material interacting with a supercritical fluid, and the third spectrometer interacting with the processed material; yet the terminology is optionally used in any order, such as to refer to a first spectrometer monitoring material interacting with the supercritical fluid, a second spectrometer monitoring the processed material, and a third spectrometer monitoring the initial material.

Generally, elements of the sensor system 900 aid the main controller 160 in: (1) enhancing efficiency of the supercritical fluid system 110, such as in terms of production, separation, and/or deriving breakdown products and/or (2) in the control, use, and/or optimization of any element linked to and/or used in the recycling system 100. Generally, any sensor element, use, and/or description in any of the above described examples is optionally used to further describe any other sensor element used herein.

### Phase Diagrams

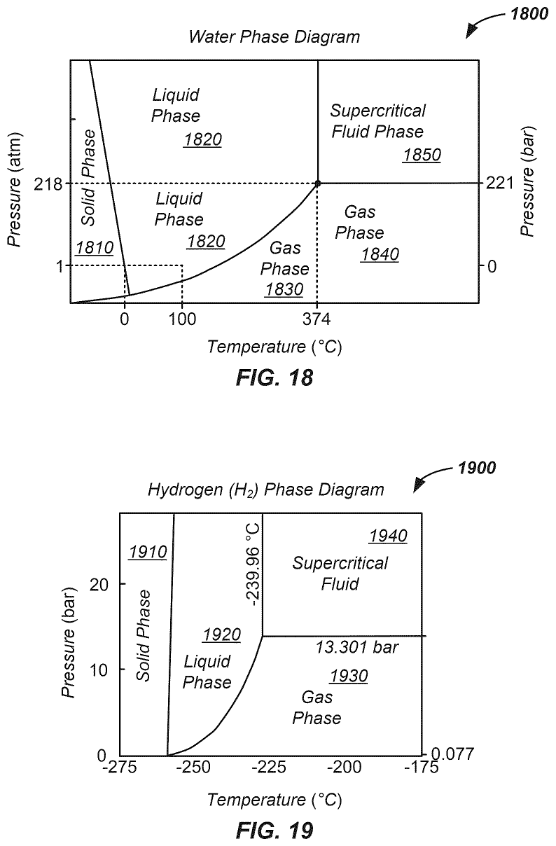

Referring now to FIGS. 18 and 19, the supercritical fluid system 110 optionally uses any supercritical fluid/supercritical fluid solvent.

In a first example, referring still to FIG. 18, the supercritical fluid is water; a water phase diagram 1800 is illustrated in FIG. 18. Generally, the recycling system 100 uses a pressure of greater than 220, 240, 300, 400, or 500 bar and/or greater than 217, 218, 250, or 300 atmospheres in the supercritical fluid chamber 112 and/or uses a temperature in excess of 373, 374, 400, or 500° C. in the supercritical fluid chamber 112.

In a second example, referring again to FIG. 19, the supercritical fluid is hydrogen/molecular hydrogen; a hydrogen phase diagram 1900 is illustrated in FIG. 19. Generally, the recycling system 100 uses a pressure of greater than 13, 13.3, 50, 100, 200, 240, 300, 400, or 500 bar, where the higher pressures form the supercritical water phase for comingled water, in the supercritical fluid chamber 112 and/or uses a temperature in excess of −240, −239, −100, 0, 100, 200, 373, 374, 400, or 500° C. in the supercritical fluid chamber 112, where the higher temperatures yield comingled water with the molecular hydrogen in a supercritical phase supercritical fluid.

### Controllers

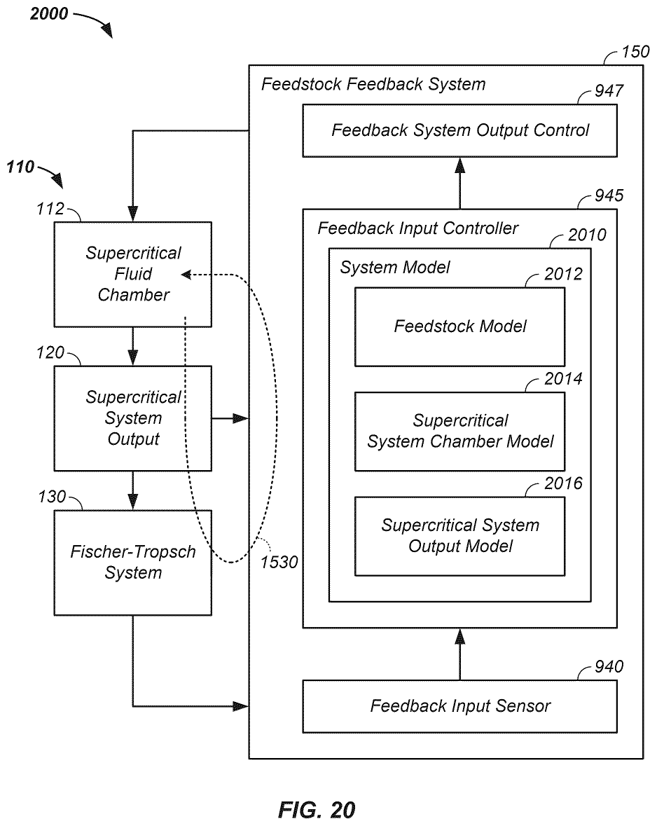

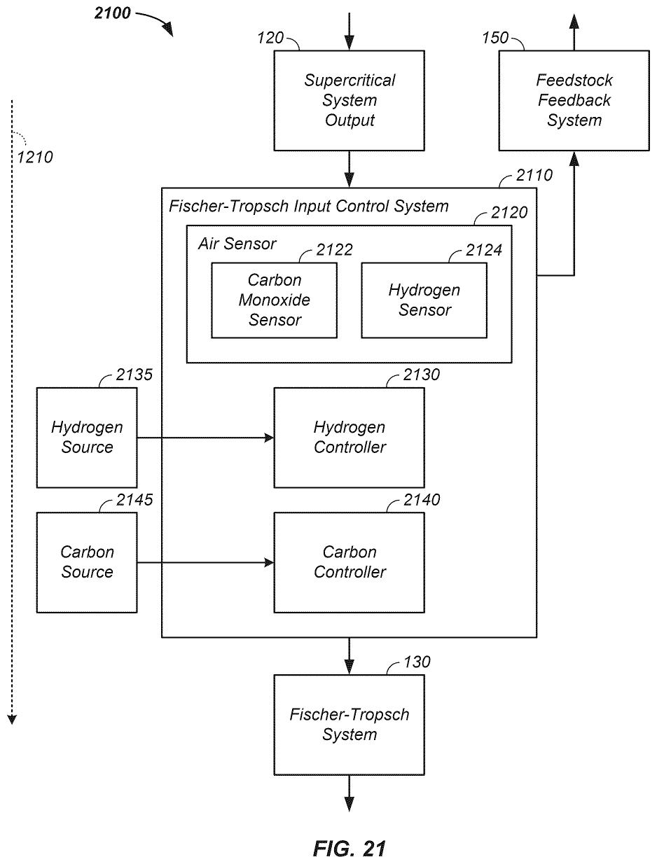

Referring now to FIG. 20 and FIG. 21, the feedback loop 1530, which optionally includes any one or more elements of the above described: hydrogen feedback system 1010, the Fischer-Tropsch feedback system 1220, and/or the high temperature water-electrolysis feedback system 1410 is further described. For clarity of presentation and without loss of generality, two examples of control loops are described.

### EXAMPLE I

Referring now to FIG. 20, a first feedback loop control system 2000, exemplary of a feedback control loop 2000, is illustrated. Generally, as described supra, feedstocks are broken down in the supercritical fluid chamber 112 of the supercritical fluid system 110 and are transferred into the Fischer-Tropsch system via the supercritical system output 130. One or more outputs of the Fischer-Tropsch system, such as described supra, are fed into the feedstock feedback system 150, described here.

Still referring to FIG. 20, the Fischer-Tropsch feedstock feedback system 150 optionally and preferably includes a feedback input sensor 940 linked to input into a feedback input controller 945, which is then linked to a feedback system output control 947, that controls output/feedback into the supercritical fluid chamber 112. Any of the sensors herein, such as the feedback input sensor optionally and preferably measure at least physical parameters of the system, such as temperature, pressure, and/or flow rate and/or chemical composition of the flow at one or more locations in the feedstock feedback system 150 linked to the Fischer-Tropsch system 130 as illustrated, such as at a feedstock feedback input location and/or in a feedstock feedback output location linked to the supercritical fluid chamber. As the illustrated feedback loop 1530 refers to any feedback system, the feedback input sensor is optionally linked to any hydrogen input, such as from the hydrogen feedback system 1010 and/or from the electrolysis system 138, described supra.

Still referring to FIG. 20, the feedback input controller 945 optionally and preferably comprises a system model 2010 including one or more of a feedstock model 2012, a supercritical system chamber model 2014, and/or a supercritical system output model 2016 and/or sensors thereof used to adjust the feedback system output control 947, which modifies parameter(s) controlling an operational aspect of the system, such as a flow rate, a temperature, a pressure, a feed rate, and/or a mixture ratio of two or more feedstocks. The feedback input controller 945 optionally and preferably includes a physics-based mathematical model of the system, that is used to forward-predict how the system will behave in response to changes in the control inputs, in any one or more of the feedstock model 2012, the supercritical system chamber model 2014, and/or the supercritical system output model 2016. The physics based models enable the feedback input controller 945 to account for the complex physics and chemistry of the system and/or time delays between changes to inputs and observing the resulting changes to the system outputs.

### EXAMPLE II

Referring now to FIG. 21, a second feedstock loop 2100, exemplary of a feedback control loop 2000, is illustrated. Generally, in the supercritical fluid Fischer-Tropsch fuel production system 1210, the above described supercritical system output 120 is optionally and preferably linked to the Fischer-Tropsch system 130 via a Fischer-Tropsch input control system 2110.

Still referring to FIG. 21, the Fischer-Tropsch input control system 2110 optionally and preferably includes an air sensor 2120 sensing at least a carbon input and a hydrogen supply from the supercritical system output 120, such as via a carbon monoxide sensor 2122 and a hydrogen sensor 2124, respectively. For clarity of presentation and without loss of generality, the air sensor 2120 is described herein using a spectrometer, such as a mid-infrared spectrometer; however, any carbon sensor in combination with any hydrogen sensor of any electrochemical and/or chemical sensor is optionally used. Further, the hydrogen sensor ultimately is used to determine a first number of hydrogen atoms, such as in any molecular form. For instance, a molecule of hydrogen gas yields two hydrogen atoms. Similarly, the carbon sensor ultimately is used to determine a second number of carbon atoms, such as in any molecular form. For instance, a molecule of carbon monoxide yields one carbon atom. In the illustrative use of a mid-infrared spectrometer, carbon monoxide is optionally sensed within ±50 nm of 4720 nm, 4670 nm, 4610 nm, 2330 nm, 1580, and/or 1568 nm and the sensed signal is used with a calibration to determine a number of moles of various carbon molecules and carbon atoms thereof. Similarly, an emission spectrometer is optionally used to measure hydrogen gas, such as with emission wavelengths within ±20 nm of 1875 nm and/or 820 nm and/or with an absorbance spectrometer at 135,000±4000 cm⁻¹.

Still referring to FIG. 21, the Fischer-Tropsch system is optionally used to produce a given fuel type, such as petroleum fuel like kerosene, a component of kerosene, a diesel fuel, and/or a component of diesel fuel, where kerosene is a lighter/more refined fuel with a lower boiling point than diesel. Herein, for clarity of presentation and without loss of generality, kerosene is used as an illustrative desired end product of the Fischer-Tropsch system 130. Generally, kerosene has a molecular formula represented as C₁₂H₂₆ to C₁₅H₃₂, which is a mixture of hydrocarbons with 12 to 15 atoms, such as in linear chains, which has a hydrogen-to-carbon ratio of 2.15±0.02. In a first case, if the air sensor 2120 senses a lower than desired hydrogen-to-carbon ratio, then an optional hydrogen controller 2130 is used to bring additional hydrogen, from a hydrogen source, into a feed source of an input stream into the Fischer-Tropsch system 130, such as to supplement hydrogen from the supercritical system output 120. The hydrogen source 2135 is optionally any source of hydrogen, but is optionally any hydrogen source described herein, such as from the electrolysis system 138 and/or the hydrogen feedback system 1010. Similarly, in a second case, if the air sensor 2120 senses a higher than desired hydrogen-to-carbon ratio, then an optional carbon controller 2140 is used to bring additional carbon, from a carbon source, into a feed source of an input stream into the Fischer-Tropsch system 130, such as to supplement carbon monoxide from the supercritical system output 120. A preferable form of carbon from the carbon source is carbon monoxide. A preferred ratio of hydrogen to carbon monoxide, fed into the Fischer-Tropsch system 130, yields 2.15 hydrogen atoms for every 1 carbon atom, but optionally the feeds are greater than 1, 1.5, 1.75, or 2 hydrogen atoms per carbon atom and/or less than 4, 3, 2.5, or 2.3 hydrogens atoms per carbon atom, where a molecule of hydrogen gas, H₂, yields two hydrogen atoms and a molecule of carbon monoxide, CO, yields one carbon atom. As illustrated, the Fischer-Tropsch input control system 2110 optionally feeds any one or more of the chemical constituents therein back into the feedstock feedback system 150.

Still referring to FIG. 21 and referring again the FIG. 1, the Fischer-Tropsch system 130 produces a fuel 140. For clarity of presentation and without loss of generality, at least one component of kerosene, kerosene, at least one component of diesel fuel, and/or diesel fuel are used as examples of the produced fuel 140. Generally, an accepted chemical formula for kerosene, a fuel, is a mix of C₁₂H₂₆ to C₁₅H₃₂, which are hydrocarbon molecules that contain carbon and hydrogen. Further, generally an accepted chemical formula for diesel fuel is a mixture of hydrocarbons ranging from C₁₀H₂₀ to C₁₅H₂₈, such as with an mean chemical formula of a twelve or thirteen carbon length chain, where the hydrocarbons are optionally and preferably composed of paraffins and aromatics. While diesel has other components, both kerosene and diesel contain saturated hydrocarbons, which are examples of a class of chemicals that are referred to as aliphatic. Aliphatics are organic compounds bound together with single bonds. Here, common aliphatics of kerosene and diesel include at least carbon chains with 12, 13, 14, and/or 15 carbons/molecular chain. An optional an preferred fuel 140 produced by the Fischer-Tropsch system 130 includes one molecules, carbon chains, and/or aliphatics with one or more of 12, 13, 14, and 15 carbons per molecule.

### Main Controller

The main controller 160/controller/system controller, a localized communication apparatus, and/or a system for communication of information optionally comprises one or more subsystems stored on a client. The client is a computing platform configured to act as a client device or other computing device, such as a computer, personal computer, a digital media device, and/or a personal digital assistant. The client comprises a processor that is optionally coupled to one or more internal or external input device, such as a mouse, a keyboard, a display device, a voice recognition system, a motion recognition system, or the like. The processor is also communicatively coupled to an output device, such as a display screen or data link to display or send data and/or processed information, respectively. In one embodiment, the communication apparatus is the processor. In another embodiment, the communication apparatus is a set of instructions stored in memory that is carried out by the processor.

The client includes a computer-readable storage medium, such as memory. The memory includes, but is not limited to, an electronic, optical, magnetic, or another storage or transmission data storage medium capable of coupling to a processor, such as a processor in communication with a touch-sensitive input device linked to computer-readable instructions. Other examples of suitable media include, for example, a flash drive, a CD-ROM, read only memory (ROM), random access memory (RAM), an application-specific integrated circuit (ASIC), a DVD, magnetic disk, an optical disk, and/or a memory chip. The processor executes a set of computer-executable program code instructions stored in the memory. The instructions may comprise code from any computer-programming language, including, for example, C originally of Bell Laboratories, C++, C #, Visual Basic® (Microsoft, Redmond, WA), Matlab® (MathWorks, Natick, MA), Java® (Oracle Corporation, Redwood City, CA), and JavaScript® (Oracle Corporation, Redwood City, CA).

The main controller/controller/system controller comprises computer implemented code to control one or more sub-systems. The computer implemented code is programmed in any language by one skilled in the art of the subsystem and/or by a skilled computer programmer appropriate to the task. Herein, for clarity of presentation and without loss of generality, specific computer code is not presented, whereas computer code appropriate to the task is readily available commercially and/or is readily coded by a computer programmer with skills appropriate to the task when provided the invention as described herein.

Herein, an element and/or object is optionally manually and/or mechanically moved, such as along a guiding element, with a motor, and/or under control of the main controller.

Still yet another embodiment includes any combination and/or permutation of any of the elements described herein.

Herein, any number, such as 1, 2, 3, 4, 5, is optionally more than the number, less than the number, or within 1, 2, 5, 10, 20, or 50 percent of the number.

The particular implementations shown and described are illustrative of the invention and its best mode and are not intended to otherwise limit the scope of the present invention in any way. Indeed, for the sake of brevity, conventional manufacturing, connection, preparation, and other functional aspects of the system may not be described in detail. Furthermore, the connecting lines shown in the various figures are intended to represent exemplary functional relationships and/or physical couplings between the various elements. Many alternative or additional functional relationships or physical connections may be present in a practical system.

In the foregoing description, the invention has been described with reference to specific exemplary embodiments; however, it will be appreciated that various modifications and changes may be made without departing from the scope of the present invention as set forth herein. The description and figures are to be regarded in an illustrative manner, rather than a restrictive one and all such modifications are intended to be included within the scope of the present invention. Accordingly, the scope of the invention should be determined by the generic embodiments described herein and their legal equivalents rather than by merely the specific examples described above. For example, the steps recited in any method or process embodiment may be executed in any order and are not limited to the explicit order presented in the specific examples. Additionally, the components and/or elements recited in any apparatus embodiment may be assembled or otherwise operationally configured in a variety of permutations to produce substantially the same result as the present invention and are accordingly not limited to the specific configuration recited in the specific examples.

Benefits, other advantages and solutions to problems have been described above with regard to particular embodiments; however, any benefit, advantage, solution to problems or any element that may cause any particular benefit, advantage or solution to occur or to become more pronounced are not to be construed as critical, required or essential features or components.

As used herein, the terms “comprises”, “comprising”, or any variation thereof, are intended to reference a non-exclusive inclusion, such that a process, method, article, composition or apparatus that comprises a list of elements does not include only those elements recited, but may also include other elements not expressly listed or inherent to such process, method, article, composition or apparatus. Other combinations and/or modifications of the above-described structures, arrangements, applications, proportions, elements, materials or components used in the practice of the present invention, in addition to those not specifically recited, may be varied or otherwise particularly adapted to specific environments, manufacturing specifications, design parameters or other operating requirements without departing from the general principles of the same.

Although the invention has been described herein with reference to certain preferred embodiments, one skilled in the art will readily appreciate that other applications may be substituted for those set forth herein without departing from the spirit and scope of the present invention. Accordingly, the invention should only be limited by the Claims included below.

1. A method for processing waste material with a supercritical fluid to produce a Fischer-Tropsch process input, comprising the steps of:
breaking down the waste material in the supercritical fluid in a supercritical fluid chamber; and
transferring into an input chamber of the Fischer-Tropsch process both carbon monoxide and molecular hydrogen formed in said step of breaking down.

2. The method of claim 1, further comprising the steps of:
incorporating at least supercritical water in the supercritical fluid; and
maintaining a pressure of greater than two hundred seventeen bar in said supercritical fluid chamber.

3. The method of claim 2, further comprising the step of:
monitoring at least one breakdown process of the waste material with light from a spectrometer penetrating into said supercritical fluid chamber.

4. The method of claim 3, further comprising the step of:
generating a fuel in a Fischer-Tropsch system with at least a portion of the carbon monoxide and at least a portion of the molecular hydrogen.

5. The method of claim 1, further comprising the step of:
moving the molecular hydrogen and the carbon monoxide from the input chamber into a Fischer-Tropsch system configured to incorporate the Fischer-Tropsch process.

6. The method of claim 5, further comprising the step of:
feeding back into said supercritical fluid chamber at least one byproduct generated in the Fischer-Tropsch system.

7. The method of claim 1, further comprising the step of:
sensing a carbon monoxide quantity that is input into said input chamber of a Fischer-Tropsch system, at least a portion of the carbon monoxide quantity comprising the carbon monoxide output from said supercritical fluid chamber.

8. The method of claim 7, said step of sensing further comprising the step of:
spectroscopically measuring interaction of light with the carbon monoxide.

9. The method of claim 7, further comprising the step of:
determining a molecular hydrogen quantity that is input into said input chamber of the Fischer-Tropsch system, at least a portion of the molecular hydrogen quantity comprising the molecular hydrogen output from said supercritical fluid chamber.

10. The method of claim 9, said step of determining further comprising the step of:
obtaining a reading from a non-spectroscopic sensor for use in determination of the molecular hydrogen quantity.

11. The method of claim 9, further comprising the step of:
incorporating into the molecular hydrogen an auxiliary hydrogen gas from a non-supercritical fluid source.

12. The method of claim 11, further comprising the step of:
electrochemically breaking apart water from said supercritical fluid chamber to form at least a portion of the auxiliary hydrogen gas.

13. The method of claim 1, further comprising the step of:
generating a fuel from at least a portion of both the carbon monoxide and the molecular hydrogen in a Fischer-Tropsch system comprising a Fischer-Tropsch reactor.

14. The method of claim 13, said step of generating further comprising the step of:
generating an aliphatic, said aliphatic comprising at least a carbon chain comprising twelve carbons.

15. The method of claim 13, said step of generating further comprising the step of:
generating a component of diesel fuel.

16. The method of claim 13, said step of generating further comprising the step of:
generating a component of kerosene.

17. A method for processing waste material with a supercritical fluid to produce a Fischer-Tropsch process input, comprising the steps of:
breaking down the waste material in the supercritical fluid in a supercritical fluid chamber;
incorporating at least supercritical water in the supercritical fluid;
maintaining a pressure of greater than two hundred seventeen bar in said supercritical fluid chamber;
monitoring at least one breakdown process of the waste material with light from a spectrometer penetrating into said supercritical fluid chamber;
spectroscopically sensing a carbon monoxide quantity that is input into said input chamber of a Fischer-Tropsch system, at least a portion of the carbon monoxide quantity comprising the carbon monoxide output from said supercritical fluid chamber, the Fischer-Tropsch system comprising the Fischer-Tropsch process;
non-spectroscopically determining a molecular hydrogen quantity that is input into said input chamber of the Fischer-Tropsch system, at least a portion of the molecular hydrogen quantity comprising the molecular hydrogen output from said supercritical fluid chamber;
transferring into an input chamber of the Fischer-Tropsch process both carbon monoxide and molecular hydrogen formed in said step of breaking down and an auxiliary hydrogen gas from a non-supercritical fluid source;
feeding back into said supercritical fluid chamber at least one byproduct generated in the Fischer-Tropsch system; and
generating a fuel in the Fischer-Tropsch system with at least a portion of the carbon monoxide and at least a portion of the molecular hydrogen, the fuel comprising a twelve carbon aliphatic component of both kerosene and diesel.
